(19) United States
(12) Patent Application Publication
(10) Pub. No.: US 2024/0249409 A1
(43) Pub. Date: Jul. 25, 2024

(54) SYSTEM AND METHOD FOR ANALYZING ABDOMINAL SCAN

(71) Applicants: Ramot at Tel-Aviv University Ltd., Tel-Aviv (IL); Sheba Impact Ltd., Ramat-Gan (IL)

(72) Inventors: Arnaldo MAYER, Ramat-Gan (IL); Tamir MANOR, Tel-Aviv (IL); Asaf HALLE, Tel-Aviv (IL); Nahum KIRYATI, Tel-Aviv (IL); Eliahu KONEN, Ramat-Gan (IL); Jana PICKOVSKY, Ramat-Gan (IL)

(73) Assignees: Ramot at Tel-Aviv University Ltd., Tel-Aviv (IL); Sheba Impact Ltd., Ramat-Gan (IL)

(21) Appl. No.: 18/561,749

(22) PCT Filed: May 18, 2022
(86) PCT No.: PCT/IL2022/050518

§ 371 Date: Nov. 17, 2023

Related U.S. Application Data
(60) Provisional application No. 63/189,769, filed on May 18, 2021.

(51) Int. Cl. G06T 7/00; G06T 7/11

(52) U.S. Cl. CPC G06T 7/0012; G06T 7/11; G06T 2207/10081; G06T 2207/20081; G06T 2207/20084; G06T 2207/30028

(57) ABSTRACT

A method of analyzing an abdominal computed tomography (CT) scan comprises applying a colon segmentation machine learning procedure to the CT scan, and receiving from the colon segmentation machine learning procedure an output indicative of a plurality of colon segments. The method also comprises feeding the output into a colon lesion detection machine learning procedure, and receiving from the colon lesion detection machine learning procedure an output indicative of presence of at least one pathology in the colon.

### RELATED APPLICATION

This application claims the benefit of priority of U.S. Provisional Patent Application No. 63/189,769 filed on 18 May 2021, the contents of which are incorporated herein by reference in their entirety.

### FIELD AND BACKGROUND OF THE INVENTION

The present invention, in some embodiments thereof, relates to the analysis of medical images and, more particularly, but not exclusively, to a system and method for analyzing an abdominal scan.

Colon cancer, also called colorectal cancer (CRC), is known as the third most common type of cancer and the second most common death cancer cause in the United States [R. L. Siegel et al., CA: A Cancer Journal for Clinicians, vol. 70, no. 3, pp. 145-164, 2020]).

Endoscopic colonoscopy is an examination technique for colorectal cancer diagnosis, and is considered a gold standard technique that can achieve high sensitivity results. Computed tomography (CT) is a powerful tool for abdominal imaging, and its common use for colon imaging, as an alternative to endoscopic colonoscopy, is known as virtual colonoscopy, also referred to in the literature as CT Colonography (CTC).

CTC allows visualization of non-invasively obtained patient-specific anatomic structures, avoiding risks, such as perforation, infection, hemorrhage, and so forth, associated with real endoscopy. CTC provides the endoscopist with important information prior to performing an actual endoscopic examination. Such understanding can minimize procedural difficulties, decrease patient morbidity, enhance training and foster a better understanding of therapeutic results

As in endoscopic colonoscopy, CTC requires patient's preparation including voiding and air insufflation of the bowels. Oftentimes, the voiding process is not optimal, leaving stool remnants in bowels, and so patient's preparation also includes administering oral contrast media, such as barium or iodinated compounds, so that the barium or iodinated compound mix with the food and enhances the signal of the stool in an attempt to make the stool differentiable from polyps. It is recognized that absent proper preparation of the patient ahead of a CTC procedure leads to a failure to detect colon cancer in about 20% of the cases [E. Klang et al., Clinical Radiology, vol. 72, no. 10, pp. 858-863, 2017].

### SUMMARY OF THE INVENTION

According to some embodiments of the invention the present invention there is provided a method of analyzing an abdominal computed tomography (CT) scan. The method comprises: applying a colon segmentation machine learning procedure to the CT scan, and receiving from the colon segmentation machine learning procedure an output indicative of a plurality of colon segments. The method also comprises feeding the output into a colon lesion detection machine learning procedure, and receiving from the colon lesion detection machine learning procedure an output indicative of presence of at least one pathology in the colon.

According to some embodiments of the invention the method comprises feeding the CT scan also into the colon lesion detection machine learning procedure.

According to some embodiments of the invention the method comprises defining a plurality of patches over the CT scan, wherein the colon segmentation machine learning procedure is applied separately to each patch.

According to some embodiments of the invention the method comprises feeding a position of each patch to the colon segmentation machine learning procedure.

According to some embodiments of the invention colon segmentation machine learning procedure comprises a convolutional neural network (CNN) having convolutional layers.

According to some embodiments of the invention the method comprises defining a plurality of patches over the CT scan, wherein the colon lesion detection machine learning procedure is applied separately to each patch.

According to some embodiments of the invention the colon lesion detection machine learning procedure comprises a convolutional neural network (CNN) having convolutional layers.

According to some embodiments of the invention the method comprises acquiring the CT scan from a subject having non-empty and un-insufflated colon.

According to some embodiments of the invention the method comprises transmitting the CT scan to a remote location, wherein the applying and the feeding is executed by a computer at the remote location.

According to an aspect of some embodiments of the present invention there is provided a computer software product. The computer software product comprises a computer-readable medium in which program instructions are stored, which instructions, when read by a computer, cause the data processor to receive an abdominal computed tomography (CT) scan and to execute the method as delineated above and optionally and preferably as further detailed below.

According to an aspect of some embodiments of the present invention there is provided a system for analyzing an abdominal computed tomography (CT) scan. The system comprises: a computer readable medium storing a trained colon segmentation machine learning procedure, and a trained colon lesion detection machine learning procedure. The system further comprises a computer having an image processing circuit configured to access the computer readable medium, to apply the colon segmentation machine learning procedure to the CT scan, to receive from the colon segmentation machine learning procedure an output indicative of a plurality of colon segments, to feed the output into the colon lesion detection machine learning procedure, and to receive from the colon lesion detection machine learning procedure an output indicative of presence of at least one pathology in the colon.

According to some embodiments of the invention the output of the colon segmentation machine learning procedure comprises a colon segmentation binary mask.

According to some embodiments of the invention the output of the colon lesion detection machine learning procedure comprises a colon lesion binary mask.

According to some embodiments of the invention the computer is configured to feed the CT scan also into the colon lesion detection machine learning procedure.

According to some embodiments of the invention the computer is configured to defining a plurality of patches over the CT scan, wherein the colon segmentation machine learning procedure is applied separately to each patch.

According to some embodiments of the invention the colon segmentation machine learning procedure comprises a convolutional neural network (CNN) having convolutional layers.

According to some embodiments of the invention the computer is configured to feed a position of each patch to the colon segmentation machine learning procedure. According to some embodiments of the invention the colon segmentation machine learning procedure comprises a convolutional neural network (CNN) having convolutional layers and a fully connected layer receiving the position together with an output from the convolutional layers.

According to some embodiments of the invention the computer is configured to define a plurality of patches over the CT scan, wherein the colon lesion detection machine learning procedure is applied separately to each patch.

According to some embodiments of the invention the colon lesion detection machine learning procedure comprises a convolutional neural network (CNN) having convolutional layers.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for of analyzing an abdominal computed tomography (CT) scan of a subject, according to some exemplary embodiments of the present invention;

FIG. 2 is a schematic illustration of a convolutional neural network (CNN) suitable for use as a colon segmentation machine learning procedure, according to some embodiments of the present invention;

FIG. 3 is a schematic illustration of a CNN suitable for use as a lesion detection machine learning procedure, according to some embodiments of the present invention;

FIG. 4 is a schematic illustration of a CT system, according to some embodiments of the present invention;

FIG. 5 is a schematic illustration of a machine learning pipeline used in experiments performed according to some embodiments of the present invention;

FIG. 6 is a schematic illustration of a CNN used in experiments performed according to some embodiments of the present invention as a colon segmentation machine learning procedure;

FIG. 7 is a schematic illustration of a noisy-student self-training flow used in experiments performed according to some embodiments of the present invention;

FIG. 8 is an abdominal CT slice superimposed with sparse annotation of colon cross-marks, as obtained in experiments performed according to some embodiments of the present 20 invention;

FIG. 9 is an abdominal CT slice superimposed with a bounding box annotation of a lesion, as obtained in experiments performed according to some embodiments of the present invention; and

FIG. 10 is a schematic illustration visualizing colon segmentations for the different methods, as obtained in experiments performed according to some embodiments of the present invention.

### DESCRIPTION OF SPECIFIC EMBODIMENTS OF THE INVENTION

The present invention, in some embodiments thereof, relates to the analysis of medical images and, more particularly, but not exclusively, to a system and method for analyzing an abdominal scan.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

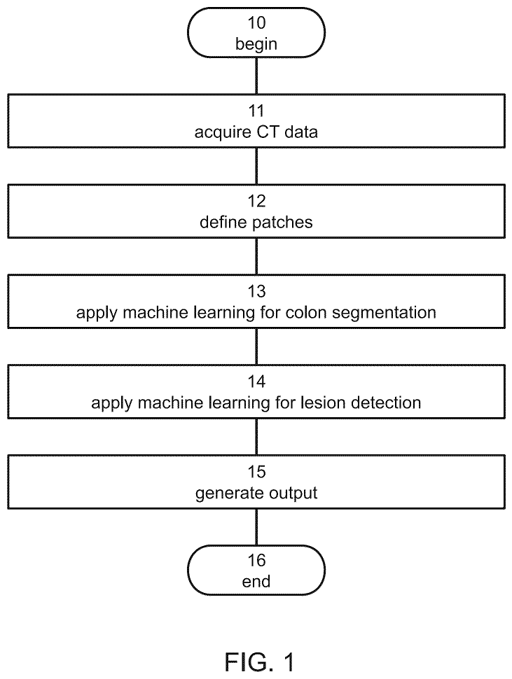

FIG. 1 is a flowchart diagram of a method suitable for of analyzing an abdominal computed tomography (CT) scan of a subject, according to various exemplary embodiments of the present invention. It is to be understood that, unless otherwise defined, the operations described herein below can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The field-of-view of the CT scan that is analyzed preferably includes at least the colon of the subject. The analysis of the CT scan can be used for detecting lesions, polyps, and/or other masses in the colon of the subject. Such a detection can aid in early-stage detection of colorectal cancer. Preferably, the CT scan is a scan that is acquired from a subject having a non-empty and un-insufflated colon. Thus, at the time of the scan, at least 50% of the volume of the colon can contain stool. For example, the CT scan can be a routine abdominal CT scan of a subject that did not perform any voiding and any insufflation of the colon.

At least part of the operations described herein can be implemented by a data processing system, e.g., a dedicated circuitry or a general purpose computer having an image processor, configured for receiving CT data and executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. In some embodiments, the method executes computer instructions of a docker that contains instructions to receive MR data and instructions to process the MR data, wherein the instructions to process the MR data include a set of instructions to generate a connectome and a set of instructions to apply machine learning. Optionally and preferably, the docker's instructions to process the MR data are executed automatically, without user intervention. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pull these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

The method begins at 10 and optionally and preferably continues to 11 at which CT data are acquired from the abdomen of the subject. The CT data can be acquired by a CT scanner configured to provide a CT scan, and can be in the form of an image or a plurality of image slices.

References to an “image” or a “scan” herein are, inter alia, references to values at picture-elements treated collectively as an array. Thus, the terms “image” and “scan” as used herein also encompasses a mathematical object which does not necessarily correspond to a physical object. The original CT scans certainly do correspond to physical objects which are the body section from which the CT scans are acquired. Each picture-element in the scan is typically associated with an digital intensity value, thus representing the scan as a grayscale image.

The acquisition of a CT scan typically includes emission of an X-Ray beam at each of several projections and detection of the beam, once attenuated by the body, by a detector array to provide a set of CT slices forming the CT scan in which each CT slice corresponds to one of the projections. The number of CT slices in a set depends on the desired field of view and the slice thickness.

The CT data can conveniently be described as a matrix. Without loss of generality, a CT scan can be denoted as I(i, j, k), where I is a digital intensity value (or a set of digital intensity values in case of a color image), (i,j) denote in-plane coordinates of picture-elements, and k is a pointer to a plane containing the picture-element at (i, j). For example, k can be a slice number of the CT scan.

Alternatively, data describing the aforementioned CT scan can be obtained from an external source (e.g., read from a computer readable storage medium, or directly from the storage of the CT scanner, or downloaded over a communication network from a cloud storage facility, or a remote computer or CT scanner), in which case 11 can be skipped.

The method optionally and preferably continues to 12 at which a plurality of patches are defined over the CT scan. The patches are preferable three-dimensional. For example, each of the patches can be defined as a set of overlapping areas across two or more slices of the CT scan. The dimensions of each patch can be from a few voxels to a few tens of voxels along each of the three orthogonal axes that define the three-dimensional patch. Representative examples of patches' size include A×B×C, where each of A, B, and C is independently from about 20 voxels to about 50 voxels. The advantage of defining patches is that it facilitates the identification of colon segments within the CT scan. As further detailed below, the method of the present embodiments employs machine learning procedure for identifying the colon within the CT scan. The inventors found that using patches, instead of the entire CT scan, significantly increases the size of the training dataset that can be used for training the machine learning procedure, since even a small number of annotated CT scans provides a large number of patches for the training dataset.

The method continues to 13 at which a colon segmentation machine learning procedure is applied to the CT scan, and an output indicative of a plurality of colon segments is received from the colon segmentation machine learning procedure. In embodiments in which patches are defined, the machine learning procedure is preferably applied separately to each patch.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, artificial neural networks (e.g., convolutional neural networks), instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

Following is an overview of some machine learning procedures suitable for the present embodiments.

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

In KNN analysis, the affinity or closeness of objects is determined. The affinity is also known as distance in a feature space between objects. Based on the determined distances, the objects are clustered and an outlier is detected. Thus, the KNN analysis is a technique to find distance-based outliers based on the distance of an object from its kth-nearest neighbors in the feature space. Specifically, each object is ranked on the basis of its distance to its kth-nearest neighbors. The farthest away object is declared the outlier. In some cases the farthest objects are declared outliers. That is, an object is an outlier with respect to parameters, such as, a k number of neighbors and a specified distance, if no more than k objects are at the specified distance or less from the object. The KNN analysis is a classification technique that uses supervised learning. An item is presented and compared to a training set with two or more classes. The item is assigned to the class that is most common amongst its k-nearest neighbors. That is, compute the distance to all the items in the training set to find the k nearest, and extract the majority class from the k and assign to item.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the likelihood for childhood obesity. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Artificial neural networks are a class of machine learning procedures based on a concept of inter-connected computer program objects referred to as neurons. In a typical artificial neural network, neurons contain data values, each of which affects the value of a connected neuron according to a pre-defined weight (also referred to as the “connection strength”), and whether the sum of connections to each particular neuron meets a pre-defined threshold. By determining proper connection strengths and threshold values (a process also referred to as training), an artificial neural network can achieve efficient recognition of patterns in data. Oftentimes, these neurons are grouped into layers. Each layer of the network may have differing numbers of neurons, and these may or may not be related to particular qualities of the input data. An artificial neural network having an architecture of multiple layer belongs to a class of artificial neural networks referred to as deep neural network.

In one implementation, called a fully-connected network, each of the neurons in a particular layer is connected to and provides input values to each of the neurons in the next layer. These input values are then summed and this sum is used as an input for an activation function (such as, but not limited to, ReLU or Sigmoid). The output of the activation function is then used as an input for the next layer of neurons. This computation continues through the various layers of the neural network, until it reaches a final layer. At this point, the output of the fully-connected network can be read from the values in the final layer.

Convolutional neural networks (CNNs) include one or more convolutional layers in which the transformation of a neuron value for the subsequent layer is generated by a convolution operation. The convolution operation includes applying a convolutional kernel (also referred to in the literature as a filter) multiple times, each time to a different patch of neurons within the layer. The kernel typically slides across the layer until all patch combinations are visited by the kernel. The output provided by the application of the kernel is referred to as an activation map of the layer. Some convolutional layers are associated with more than one kernel. In these cases, each kernel is applied separately, and the convolutional layer is said to provide a stack of activation maps, one activation map for each kernel. Such a stack is oftentimes described mathematically as an object having D+1 dimensions, where D is the number of lateral dimensions of each of the activation maps. The additional dimension is oftentimes referred to as the depth of the convolutional layer. For example, in CNNs that are configured to process two-dimensional image data, a convolutional layer that receives the two-dimensional image data provides a three-dimensional output, with two-dimensional activation maps and one depth dimension.

The advantage of using CNN is the use of layers of kernels providing the ability to learn different levels of complexity of visual aspects of the input features.

The colon segmentation machine learning procedure employed at 13 is preferably a deep neural network, more preferably a CNN.

The machine learning procedure can be trained according to some embodiments of the present invention by feeding a machine learning training program with training data. The training data includes annotated CT scans or, more preferably, annotated CT scan patches from a cohort of subjects. The annotation labels each voxel or group of voxels in the training data as either belonging to the colon or to the background of the CT scan. Once the data are fed, the machine learning training program generates a trained machine learning procedure which can then be used without the need to re-train it.

For example, when the machine learning procedure is a CNN, it can be trained according to some embodiments of the present invention by feeding a CNN training program with the training data. The training process adjusts convolutional kernels, bias matrices and other parameters of the CNN so as to produce an output that classifies each voxel or group of voxels of the CT scan or patch as close as possible to its label. The final result of the training is a trained CNN having an input layer, at least one, more preferably a plurality of, hidden layers, and an output layer, with adjusted weights assigned to each component (neuron, layer, kernel, etc.) of the network. The CNN training program thus generates a trained CNN which can then be used without the need to re-train it. A representative example of a training of a colon segmentation machine learning procedure for the case in which the machine learning procedure is a CNN is provided in the Examples section that follows.

Following the training, a validation process may optionally and preferably be applied to the trained CNN, by feeding validation data into the network. The validation data is typically of similar type as the training data, except that only the CT scans are fed to the trained network, but not their labels. The labels are used for validation by comparing the output of the trained CNN to the labels.

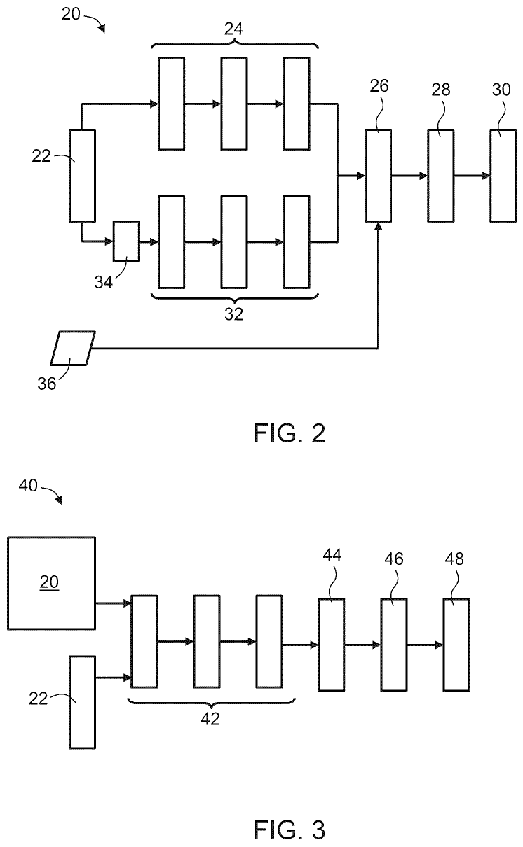

A representative example of a CNN 20 suitable for use as the colon segmentation machine learning procedure is schematically illustrated in FIG. 2. The input that is fed to CNN 20 is shown at 22. The input 22 can be the CT scan or a patch thereof. CNN 20 comprises a first set of convolutional layers 24 which receive the input 22, and a concatenator 26 that receives the output of the last convolutional layer of layers 24 and concatenates the activation maps generated by layers 24, to provide a feature vector that is preferably one-dimensional. CNN 20 also comprises a fully connected layer 28 which receives the feature vector from concatenator 26. Fully connected layer 28 typically employs a nonlinear activation function, such as, but not limited to, a sigmoid or the like, and provides a classification score which classifies the input 22. Typically the CNN 20 is trained to classify the input 22, as either a background patch or a patch that describes a region in the colon. Optionally and preferably the CNN 20 is trained to further classify non-background patches according to the anatomical segment of the colon that the patch describes. As a representative example, which is not intended to be limiting. CNN 20 can be trained to identify whether the patch is part of the ascending colon, the transverse colon, the descending colon, the sigmoid colon, of the rectum. CNN 20 can also include an output layer 30 that stores output values indicative of the classification.

In some embodiments of the present invention CNN 20 is a branched CNN, which comprises a second set of convolutional layers 32 which execute machine learning processing independently of layers 24. In these embodiments, CNN 20 preferable comprises a cropper 34 which receives the input 22, and provides a cropped version thereof. For example, cropper 34 can remove from input 22 voxels that are outside a region which contains the centroid of input 22 and which has a predetermined size. The cropped version of input 22 is fed to layers 32 and the concatenator receives also the output from the last layer of layers 32, so that the feature vector provided by concatenator 26 includes the activation maps generated by both layers 24 and 32.

The inventors found that in cases in which input 22 is a patch of the CT scan, the predictability of CNN 20 is significantly improved when the position of the patch within the scan is fed into the CNN. In these embodiments, CNN 20 comprises a patch position input layer 36 that receives the position of the patch. The position can be a tuple of coordinates in the coordinate system of the CT scan, for example, along the axes of the scanner that acquired the CT scan. The position is preferably fed to concatenator 26, so that the feature vector provided by concatenator 26 includes the activation maps generated by layers 24 and 32, and also the position of the patch.

In some embodiments of the present invention the output from layer 30 of CNN is processed. For example, the method can aggregate all the patches that are classified as describing a region of the colon, according to their position in the CT scan, so as to form a heat map of the colon describing the classification score of each patch. Also contemplated, are embodiments in which the heat map is binarized, for example, using a thresholding procedure, to transform the heat map into a colon segmentation binary mask. The colon segmentation binary mask is optionally and preferably in the form of a chain of patches, each being classified as describing a portion of the colon. Preferably, the width of the chain equals the width of a single patch and is therefore predetermined.

Referring again to FIG. 1, the method continues to 14 at which the output of the colon segmentation machine learning procedure (or a processed version of this output, e.g., a heat map or a binary mask) is fed into a colon lesion detection machine learning procedure. The colon lesion detection machine learning procedure is preferably also fed with the CT scan itself, or, with CT scan patches in embodiments in which patches of the CT scan are defined. It is not necessary for operations 13 and 14 to be applied to patches of the same size, although such a scenario is contemplated in some embodiments of the present invention.

The colon lesion detection machine learning procedure is trained to use the information in the output of the colon segmentation machine learning procedure in order to determine, for each region in the CT scan that has been segmented as a part of the colon, whether the region contains one or more lesions. When the output of the colon segmentation machine learning procedure is provided as a binary mask, the colon lesion detection machine learning procedure can mask the CT scan using the binary mask, and determine the presence or absence of lesion only in regions which are masked by the binary mask.

The colon lesion detection machine learning procedure can be of any of the aforementioned types of machine learning procedures. Preferably, the colon lesion detection machine learning procedure employed at 14 is a deep neural network, more preferably a CNN.

The colon lesion detection machine learning procedure can be trained according to some embodiments of the present invention by feeding a machine learning training program with training data. The training data used for training the colon lesion detection machine learning procedure is different than the training data used for training the aforementioned colon segmentation machine learning procedure. The training data used for training the colon lesion detection machine learning procedure is in the form of CT scans or CT scan patches, each being labeled as either having or not having some pathology (e.g., a lesion) in the colon. Preferably, the training data is based on CT scans that are labeled as characterizing subjects that have been diagnosed with the pathology, as well as on CT scans that are labeled as characterizing control subjects (e.g., healthy subjects). The training data is preferably labeled on a per voxel or per group of voxels basis, wherein each voxel or group of voxels is labeled as either describing a pathology or describing a healthy tissue.

Once the data are fed, the machine learning training program generates a trained machine learning procedure which can then be used without the need to re-train it. A representative example of a training of a colon lesion detection machine learning procedure for the case in which the machine learning procedure is a CNN is provided in the Examples section that follows.

A representative example of a CNN 40 suitable for use as the colon lesion detection machine learning procedure is illustrated in FIG. 3. The input that is fed to CNN 40 includes the output of CNN 20, or a processed version thereof and optionally and preferably also the input 22 itself, where the input 22 can be, as stated, the CT scan or a CT scan patch. CNN 40 comprises a set of convolutional layers 42 which receive the input 22 as well as the output of CNN 20, a concatenator 44 that receives the output of the last convolutional layer of layers 42 and concatenates the activation maps generated by layers 42, and a fully connected layer 46 which receives the features from concatenator 44 and provides a classification score which classifies each voxel or group of voxels in the input 22. CNN 40 can also include an output layer 48 that stores output values indicative of the classification of each voxel or group of voxels in the input 22. The classification that is stored in layer 48 is preferably binary, wherein each voxel or group of voxels is classified as either belonging to a pathology (e.g., a lesion) or not belonging to a pathology (e.g., healthy tissue).

In some embodiments of the present invention the output from layer 48 of CNN 40 is processed. For example, at 15 the method can generate a colon lesion binary mask which includes the colon segmentation binary mask on which voxels or groups of voxels are highlighted according to their binary classification. For example, a color output can be generated in which voxels or groups of voxels classified as belonging to the pathology are presented in one color and voxels or groups of voxels classified as not belonging to the pathology are presented in another color.

The method ends at 16.

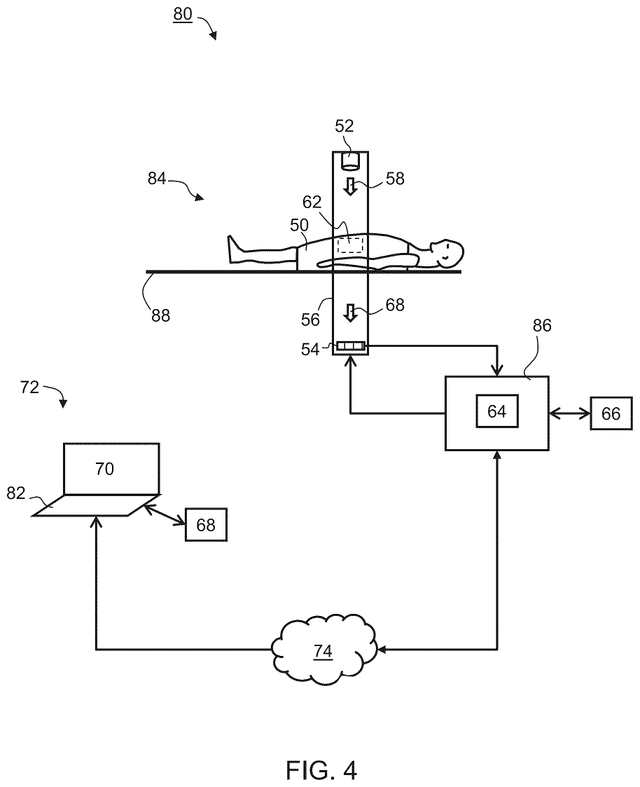

FIG. 4 is a schematic illustration of a CT system 80, according to some embodiments of the present invention. CT system 80 comprises an image processor 82, a CT scanner 84 and a computerized controller 86.

CT scanner 84 comprises a bed 88 for supporting a subject 50, an x-ray source 52 producing a collimated x-ray beam 58, and a detector array 54, configured for detecting an attenuated x-ray beam 60 formed by the interaction of beam 58 with a body region 62 of subject 50 and responsively generating an electrical signal, such as a video signal. Body region 62 preferably includes the abdomen of subject 50. Source 52 and detector 54 are mounted on an annular gantry 56 at opposite sides of the bed 88. Gantry 56 controls the position of detector 54 and source 52, and is configured to rotate around bed 88 together with detector 54 and source 52, so as to scan the direction of ray beam 58.

It is expected that during the life of a patent maturing from this application many relevant CT scanners will be developed and the scope of the term CT scanners is intended to include all such new technologies a priori.

CT scanner 84 is controlled by computerized controller 86. The signal provided by detector 54 at each projection of gantry 56 is communicated to controller 86. Computerized controller 86 has image capture hardware 64, configured to collect the signal for each projection, to digitize the signals, and to calculate from the digitized signals a tomogram of the abdomen 62, thereby providing an abdominal CT scan. In some embodiments of the present invention controller 86 is associated with a memory medium 66, preferably a non-transitory medium, which stores computer programs for calculating the tomogram, and which may also store the slices of CT scan, once generated, e.g., in the form of a plurality of images. Controller 86 also controls the angular position of gantry 88, and the operation of source 52.

Processor 82 can be local or remote with respect to scanner 84. The I/O circuits (not shown) of controller 86 and processor 82 can communicate information with each other via a wired or wireless communication. For example, controller 86 and processor 82 can communicate via a network 74, such as a direct cable, local area network (LAN), a wide area network (WAN) or the Internet. In some embodiments of the present invention processor 82 is a component in a server computer 72 that is remote from CT scanner 84. For example, server computer 72 can in some embodiments be a part of a cloud computing resource of a cloud computing facility in communication with controller 86 over network 74.

Data describing the CT scans are communicated from controller 86 to image processor 82. Processor 82 is associated with a computer readable storage medium 68 tangibly embodying a program of instructions executable by processor 82 to receive the data, to apply the colon segmentation machine learning procedure (e.g., CNN 20) to the CT scan, to receive from the colon segmentation machine learning procedure an output indicative of a plurality of colon segments, to feed the output into a colon lesion detection machine learning procedure (e.g., CNN 40), and to receive from the colon lesion detection machine learning procedure an output indicative of presence of at least one pathology (e.g., lesion) in the colon.

Processor 82 preferably displays the output from the colon lesion detection machine learning procedure or some processed version thereof (e.g., a binary mask as further detailed hereinabove) in a manner that identifies voxels or groups of voxels that describe the pathology. Display 70 can be located locally with respect to processor 82, as shown in FIG. 4, or at a location that is remote from processor 82 (e.g., near scanner 84, at an oncologist's clinic, etc.). Also contemplated, are embodiments in which display 70 is a GUI of a mobile device such as a smartphone, a tablet, a smartwatch and the like. When display 70 is remote from processor 82 or a display of a mobile device, data describing the output from the colon lesion detection machine learning procedure is transmitted over network 74 to display 70.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Neural Incidental Detection of Colon Lesions in Routine Abdominal CT

Lesions of the colon are often visible in routine abdominal CT, without any patient preparation. These incidental findings must be reported, potentially saving patients' lives. Recent studies have shown that over 20% of cancerous colon lesions are actually missed in routine abdominal CT examinations. This Example addresses this problem and present Colon-Lesion-Network (CoLesioNet), a convolutional neural network (CNN) approach for the incidental detection of suspicious colon lesions in routine abdominal CT. The CoLesioNet of the present embodiments splits the lesion detection task into two consecutive steps: 1) Colon segmentation; 2) Lesion detection within the colon segmentation obtained in the previous step. Each step relies on a dedicated 3D patch-based CNN framework. The method in this example was validated quantitatively, providing a Dice score of about 71.9% for the colon segmentation task, and an average sensitivity score of about 71.4% in the lesion detection task (CoLesioNet full pipeline). In comparative experiments, the CoLesioNet of the present embodiments outperformed conventional methods (U-Net, V-Net, nnU-Net). Considering the limited number of available ground-truth lesions, a semi-supervised learning (SSL) scheme, based on the noisy-student algorithm, is implemented in this Example to improve colon lesion detection. Thus, by incorporating unlabeled data, the semi-supervised approach of the present embodiments further improved the average sensitivity by 5% over the fully-supervised detection baseline (CoLesioNet), resulting in an average sensitivity score of 76.4%. These results suggest that the method of the present embodiments may become a valuable tool for reducing false negatives in incidental colon cancer detection using abdominal CT.

### I Introduction

According to the American Cancer Society [1], colon cancer, often called colorectal cancer (CRC), is the third most common type of cancer and the second most com-mon death cancer cause in the United States. Approximately 147,950 cases were estimated to be diagnosed with CRC, and 53,200 were predicted to die from the disease during 2020 [1]. Yet, early-stage detection of CRC significantly increases the 5-year survival rate. [2], [3].

Computed tomography is a widespread imaging modality, fast, noninvasive, accurate and highly-available [9]. Abdominal CT screening is not used for general CRC screening due to the radiation exposure. However, detecting colorectal cancer as an incidental finding in routine abdominal CT is a basic requirement. In this challenging task, wherein the bowel has not been voided and washed beforehand, a study has shown that “colon cancer is undetected in 20% of abdominal CT examinations in patients subsequently proven to have colon cancer at colonoscopy” [10].

Nowadays, deep learning techniques, usually based on CNNs, often outperform classical methods in various medical image processing tasks such as organ/colon lesion detection, classification and detection [11], [12]. Most deep-learning applications designed for image-based colorectal cancer detection rely on colonoscopy or CT colonography. A comprehensive survey recently reviewed deep learning methods for colon cancer diagnosis in 135 academic papers. None of them used routine abdominal CT as the input imaging modality [13].

Using deep learning or any other methodology, CRC diagnosis based on abdominal CT examination is challenging. The abdominal scan is a large three-dimensional volume and includes many organs (liver, lungs, spleen, etc.) that have no relation to the CRC diagnosis objective. The colon is twisted and traversed in a large area of the scan, and its shape varies between patients. Furthermore, colon lesions may differ by shape, brightness, size, and location. All of these difficulties, along with varied CT protocols (drinking/contrast materials), may lead to overfitting and poor generalization when using a small dataset.

The performance of deep learning models is dependent on the availability of an annotated dataset. Large annotated medical image datasets are costly, especially 3D volumetric abdominal scans, because both time and expertise are required to produce accurate annotations. To overcome this limitation, many methods adopted the recent progress in semi-supervised learning (SSL). In SSL, a large quantity of unlabeled data is incorporated along with the labeled data to further improve the performance of the fully-supervised baseline. A few works utilized SSL methods for medical imaging [24-26].

This Example describes CoLesioNet, a two-step patch-based framework for the automatic detection of colorectal cancer lesions in the setting of routine abdominal CT. A patch-based colon segmentation method generates a colon mask defining the search area for colon lesions. Colon patches are then processed by CoLesioNet to detect colorectal lesions. The supervised detection performance of the present embodiments was further improved by utilizing a large unlabeled colon lesion dataset using a noisy-student framework. Via a self-training strategy, a teacher model is trained on the labeled data, producing pseudo-labels from the unlabeled data. Then, a student model is trained using both labeled and pseudo-labeled examples, while infusing noise into the student model to facilitate enhanced generalization.

The method of the present embodiments is evaluated using a unique dataset, which includes a sparsely annotated colon dataset and a lesion dataset. By extensive experiments, the Inventors demonstrate that the method of the present embodiments outperforms other training strategies and methods.

### II Methods

### A. ColesioNet Architecture

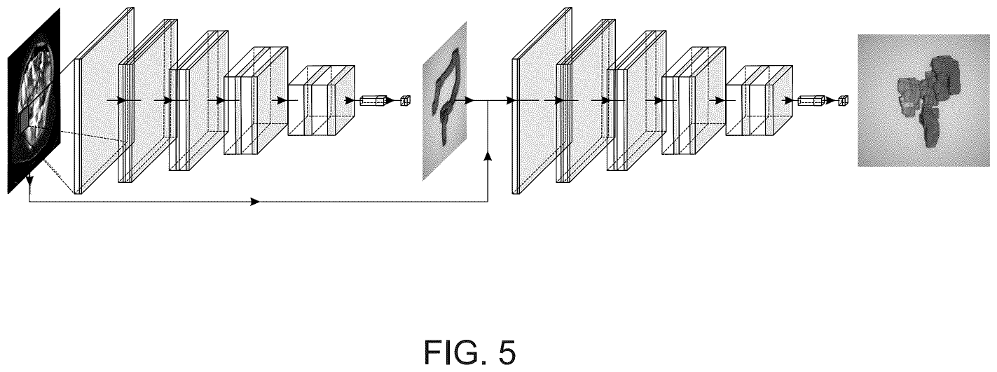

1) Two Phase Network: The CoLesioNet dual-phase framework is composed of two CNNs, one for colon segmentation and the other for colon lesion detection. The method employs patch-based approach to improve convergence and alleviate overfitting. The patch-based approach is advantageous since the colon traverses the abdominal cavity. This allows the colon to be divided into a set of small patches, each containing a colon or colonic lesion. The ColesioNet pipeline used in this Example is shown in FIG. 5. As shown, the ColesioNet network receives as input an abdominal CT volume, and produces a segmentation mask of the colon (middle image). In the second phase, the lesion network receives as input both the colon segmentation mask and the original abdominal CT volume and outputs a lesion segmentation binary mask (rightmost image—lesion voxels highlighted over colon voxels in red). Each phase applies a separate patch dataset for training. During inference, the output mask is constructed by aggregating the patch results for the given patient's volume.

Both ColesioNet phases are trained as binary classifiers, where each phase is trained on a different patch dataset. During inference, 3D abdominal patches are extracted from the input volume by a sliding window and fed to the colon classification network. The resulting scores from all patches are fed into a mask-assembling module that aggregates the patches and generates the final colon segmentation mask. The second phase yields a binary mask for lesion detection. Since patches relevant for lesion detection reside only in the colon, by using the colon segmentation mask from the previous phase, only patches predicted to be within the colon are fed into the classifier. Patches are aggregated in a similar way to the preceding phase to construct the final lesion mask.

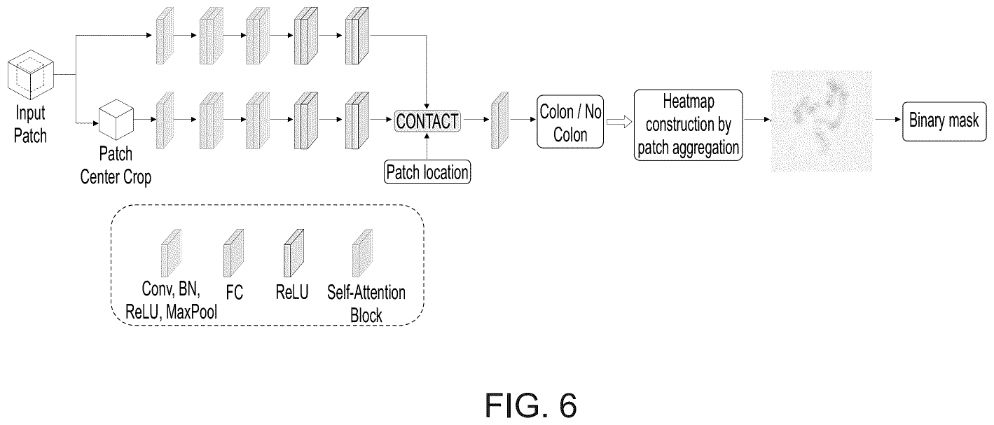

2) Colon Patch-Based Segmentation Phase: The architecture of the colon segmentation phase is shown in FIG. 6. Given an input 3D patch, both the patch and the cropped version of the patch are fed into two separate network branches. The two extracted feature maps are then combined with the patch coordinates through concatenation. The network is trained via a cross-entropy loss using the patch colon score outputted by the final fully connected layer. On inference, a colon heatmap is constructed based on the patch results and threshold it to obtain the final colon binary mask.

Given an isotropic abdominal volume x∈R^{D×H×W} where D is the number of slices, the colon segmentation network outputs a binary mask Y∈[0,1], with the value 1 indicating a colon voxel. The CNN has a multi-scale architecture consisting of two parallel branches, each branch operating on a different scale of the input patch. To capture both global spatial information and fine colon features, two different patch scales were chosen and processed in parallel convolutional branches. In FIG. 6, the upper branch extracts features from the original input patch, while the bottom branch extracts features from a center-cropped version of the same patch.

By taking advantage of the colon center-line placement within the abdominal cavity, the patch center coordinates are used as additional spatial geometry information. The coordinates are normalized to the range [0,1] where the z coordinate is measured relative to the calculated limits of the abdominal z-axis. The upper slice limit is determined by the last slice of the lung and the lower slice limit is the last slice of the scan. The two feature vectors from both branches and the 3D patch coordinates are concatenated. The resulting feature vector is fed into a fully-connected layer which classifies the patch. Each branch consists of three convolutional blocks followed by two fully-connected layers for feature extraction. The numbers of channels assigned to the three convolutional blocks are 32, 64, 128. The last two convolutional blocks are stacked with a self-attention mechanism block. Self-attention has been recently proposed as a mechanism for modeling long-range dependencies in features. It enables the network to focus on strongly related discriminative areas of the input regardless of spatial proximity. In medical image analysis, attention has been widely used as well [29-31]. In this Example the attention module presented in SAGAN was used.

The feature map of the preceding convolutional layer×E RCN (where N is the product of the spatial dimensions and C is the number of input channels) is fed into the attention block, resulting in an Attention map, an attentive version of the input features. The output is obtained by adding the input feature map to the product of the self-attention feature map and y, which is a learnable scale parameter:

$$Out = γ \cdot AttentionMap + x \qquad ( EQ . 1 )$$

3) Lesion Patch-Based Phase: Given an isotropic abdominal volume x∈R^{D×H×W} and the corresponding colon mask Se[0,1]D×H×W, the lesion phase outputs a lesion binary mask Y∈[0,1]^{D×H×W}. D being the number of slices in the CT volume, and H, W are the spatial dimensions of the input. Based on the estimated colon segmentation mask S, and the corresponding ground-truth lesion mask, the method can determine which pixels in the image volume belong to the colon, lesion, or background. Specifically, a patch dataset was generated by cutting patches out of these volumes, resulting in colon and lesion examples for 3D patch classification.

The network consists of stacked residual blocks optimized for the input patch size. The number of channels assigned to each block is 8, 16, 32, 64. Subsequently, Global Average Pooling (GAP) is applied to generate a 1×1 feature map for each corresponding channel, thus reducing the size of the preceding layer to 64×1×1. Dropout is performed before feeding the feature vector into a fully-connected layer with single logit output. The sigmoid activation operation produces the final probability score for the given patch. Each block consists of 3×3×3 convolutional kernels, where each convolution operation is followed by 3D batch normalization and ReLU activation. The last three residual blocks reduce the feature map's spatial dimensions along the network using convolution with stride 2.

### B. Semi-Supervised Lesion Detection Through Self-Training

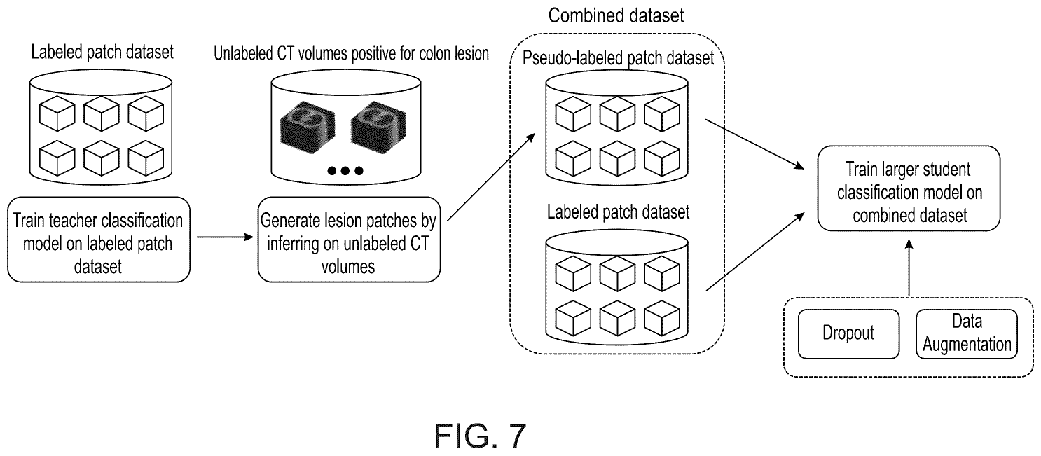

The colon lesion detection phase of the present embodiments is guided by a semi-supervised learning approach. In this Example, the Noisy-Student self-training strategy was used as part of the lesion patch-based classification network. The objective was to create a large number of labeled positive patches from a large set of non-annotated routine abdomen CT scans, containing 550 colon lesions (each from a different patient) reported by diagnostic radiologists. The training process involves three stages: first, a teacher model is trained in a supervised manner on the labeled patches data. Afterward, the teacher model produces pseudo-labels on unlabeled data by the following steps: produce colon segmentation masks from the unlabeled data, generate multiple patches along the colon, extract pseudo-labels on patches, and filter patches with low probability confidence to reduce labeling error. Then, a student model is trained using both labeled and pseudo-labeled patches. The training strategy is illustrated in FIG. 7.

The student model is enhanced by adding noise to it, as well as by training a larger model (or an equal one) than its teacher. As a result, the student model can generalize more effectively than the teacher. In this Example, data augmentation and dropout are used as noise factors, and employ a student model with double the number of channels is used at each layer. A predefined probability score threshold was use a to filter out low confidence patches, to increase the likelihood of finding relevant positive lesion patches.

### C. Patch Dataset Generation

Small datasets can lead to poor generalization results, especially in the case of 3D medical images. To overcome the shortage of annotated data, each scan is treated as a collection of patches, which produces a much larger set of examples. The colon dataset is a collection of CT volumes with corresponding pseudo ground-truth masks (after the annotation process described below). As the task is 3D patch classification, the method creates a large number of training examples per volume. For each volume, by scanning in a sliding window manner, patches that highly overlap the colon mask are regarded as positive patches, whereas negative patches are generated from regions that do not overlap with the colon mask.

Using the same approach, the lesion patch dataset was constructed. For each lesion volume, the colon mask is produced by the pre-trained colon segmentation phase. Patches indicated as part of the colon by the colon mask but do not overlap with the ground-truth lesion mask are considered as negative example patches. Patches that substantially overlap with the ground-truth lesion map are considered as positive example patches. Patches overlap with each other, resulting in generating multiple positive patches per a single lesion, thus increasing the effective training set size. The colon-phase patch size was set in this Example to 35×35×35 and the lesion-phase patch size was set in this Example to 30×40×40. As some scans are chest-abdomen CTs (and not abdominal CTs only), it is advantageous to reduce the number of slices to only those relevant to the abdomen. Thus, a classic computer vision techniques (HU thresholding, connected components, etc.) is applied on the raw scan to determine the last slice where the lung appears, allowing to sample during training and inference only patches from the abdomen ROI.

### D. Mask Assembly

Following is a description of the post-processing steps, executed during inference, which construct a mask by aggregating the patch probability scores. In the colon-phase post-processing, each patch has a probability score at the network output. Patches with high probability scores are referred to as hits. A heatmap is constructed by incrementing by 1 all the voxels that lie inside each of the 3D hit patches. Heatmap thresholding is then applied to produce the final binary colon mask. In the lesion-phase post-processing, only the thresholded lesion probability map was used to obtain the binary mask (without heatmap construction). To obtain the final lesion detection results, the output binary mask was further manipulated. Specifically, connected-components within the binary colon lesion detection mask were identified to distinguish between different candidate detections. These connected lesion components are used for lesion evaluation, as described in greater detail below.

### III Experiments and Results

### A. Data

1) Colon Dataset: Obtaining annotated medical data requires extensive radiological expertise. In particular, manual pixel-wise segmentation of the colon demands a substantial effort. Some of the difficulties are an uncleansed colon, and a large three-dimensional volumetric organ that extends throughout the abdominal cavity. Moreover, the colon content is extremely variable in unprepared patients undergoing routine abdominal CT, as opposed to CT colonography where the colon's internal voxels are mostly air-filled, facilitating the annotation process.

The Inventors collected a unique sparsely annotated dataset of 33 axial 3D abdominal CT scans. By leveraging the colon structure, the Inventors were able to label the colon region in a time-efficient manner and to construct a colon pseudo ground-truth (GT) mask as will now be explained.

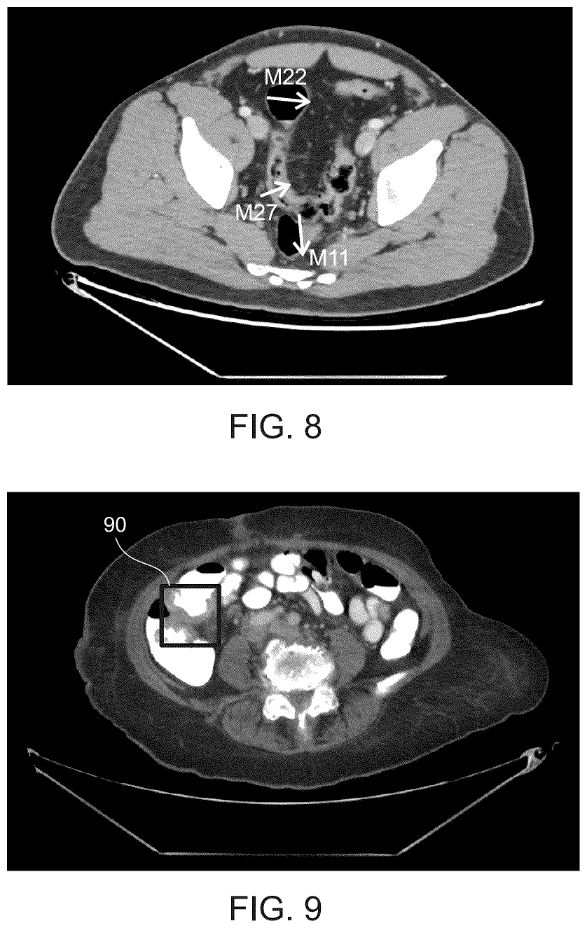

FIG. 8 shows sparse annotation of colon cross-marks on an abdominal CT slice. An experienced radiologist placed a series of marks (shown in FIG. 8 as arrow and symbols M22, M27, M11) from the rectum to the cecum, covering the entire colon, allowing the entire colon mask to be approximated. From the radiologist's annotations, point pairs in the form (3D point, symbol) were collected, and a pseudo colon-centerline was reconstructed by connecting the center points of consecutive annotations. To obtain the final pseudo ground-truth segmentation map, the centerline point set were dilated by a cubical structuring element. This labeling technique requires much less labeling time and effort by the radiologist, in contrast to the time-consuming and expensive process of fully annotating a 3D abdominal volume in a pixel-wise manner.

2) Colon Lesion Dataset: the colon lesion dataset consisted 50 3D axial abdominal CT scans. On each slice that contains a lesion, a bounding box was annotated by an expert radiologist, as shown by a black rectangle 90 in FIG. 9. For the SSL training, a dataset of 550 unlabeled scans was collected. Based on the radiologist's diagnosis, there was a strong suspicion of colon lesion in these scans. The dataset was diverse and contains a variety of shapes, lesion locations, and lesion sizes. Moreover, the dataset was not restricted to a particular scan protocol, such as drinking or intravenous contrast agent. The spatial size of each image was 512×512, and the number of slices varies from about 122 to about 386 (mean 209, standard deviation 54.22). In pre-processing, all images were resampled to an isotropic resolution of 1.0×1.0×1.0 mm. The image was clipped using a soft-tissue CT window range of [−140, 260] HU, followed by min-max normalization.

### B. Implementation

Following is a description of the training settings and the implementation environment. The CoLesioNet of the present embodiments was trained via a binary cross-entropy loss, whereas the segmentation networks used for comparison were trained using the Dice Similarity Coefficient (DSC) loss function. The Adam optimizer was used for training with a learning rate of 1×10⁴, and the network's weights were randomly initialized. For the colon segmentation phase, the data were augmented by random-axis flipping. For the lesion detection phase, data augmentations (random affine transformation) was applied to avoid overfitting. This was advantageous since the lesion dataset was small. The transformation consisted of horizontal flipping, scaling (0, ±20%) and rotation)(0, ±90°. A batch size of 32 was used a while training. For the comparative experiments, several segmentation models (including extensive data augmentation) were trained. For the 3D variants of the methods a batch size of 1 was used, due to GPU memory constraints caused by large input volumes. For the 2D variant a batch size of 32 was used.

Imbalance in the datasets was addressed as follows. In the colon patch dataset, the majority class (non-colon negative class) was down-sampled. In the colon lesion patch dataset, the number of lesion patches were balanced across cases so that each lesion will equally contribute to the training. Then the minority class samples (lesion positive class) were over-sampled to the same number of samples as the majority class.

Lesion detection evaluations were conducted using a stratified 20-fold cross-validation framework. Overall, the dataset consists of 50 positive cases (with lesion) and 50 negative cases (without lesion). The implementation was based on the PyTorch framework and used an NVIDIA RTX 2070 GPU.

### C. Metrics

For colon segmentation evaluation, the standard Dice coefficient (Dice) was used. For lesion detection, a free-response receiver operating characteristic curve (FROC) was employed [36-38]. This allowed exploring trade-offs between various operating points. Sensitivity was measured over different rates of false positive (FPs) per volume, providing various clinically meaningful operating thresholds that illustrate the recall/precision trade-off. To measure detection performance, the average sensitivity score was defined as the average of the sensitivity at four ratios of FPs per volume: 1/2, 1, 2,4.

To achieve a clinically useful result, it is typically desired to have high sensitivity while maintaining relatively low FPs/volume ratio on average. Even with perfect sensitivity, a large FPs/volume ratio reduces the benefits of an automatic detection system, as the radiologist's labor to examine the results (mostly FPs) becomes a burden.

### D. Results on Colon Segmentation

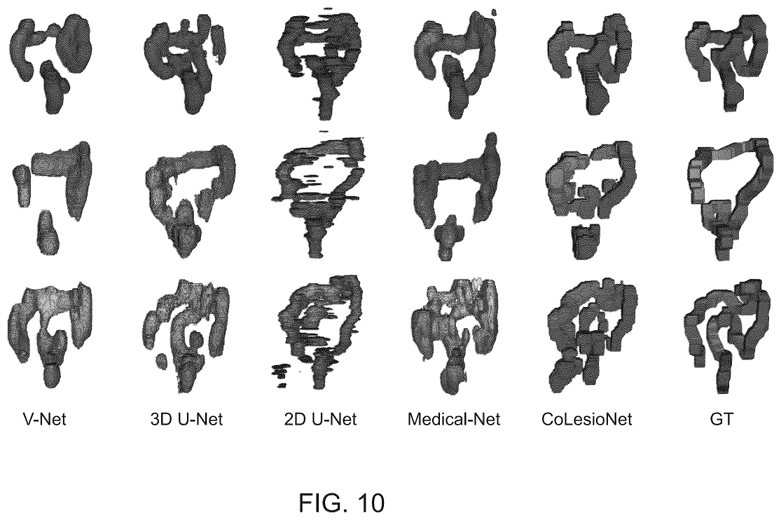

The method of the present embodiments was evaluated and compared to several recent 3D segmentation methods, including V-Net [14], 3D U-Net [15], and 3D-ResNet18 pre-trained on the MedicalNet dataset (a large dataset covering a wide range of modalities, target organs, and pathologies). In view of the data scarcity, a 2D U-Net variant was also examined. Table 1, below, and FIG. 10. The segmentation performances were evaluated using the Dice score and the Sensitivity and Precision were calculated. For the competing segmentation methods, the input volume was resampled to 2.5×1.0×1.0 mm and resized to 128×256×256, then the rest of the pre-processing steps were performed as according to the patch-based approach of the present embodiments. The results show that the method of the present embodiments outperforms all the other methods in terms of the Dice score (71.90%), while achieving competitive results on sensitivity and precision.

TABLE 1

| Method | Dice | Sensitivity | Precision |
|---|---|---|---|
| V-Net | 0.634 | 0.671 | 0.607 |
| 3D-ResNet18 | 0.663 | 0.698 | 0.635 |
| (pre-trained) | | | |
| 3D U-Net | 0.672 | 0.721 | 0.641 |
| 2D U-Net | 0.699 | 0.706 | 0.695 |
| CoLesioNet | 0.719 | 0.789 | 0.675 |

### E. Colon Lesion Detection Results

The CoLesioNet was evaluated by comparison to recent conventional approaches for 3D lesion localization, including the nnU-Net method [22]. A few of the compared methods require prior colon segmentation. For fair comparison, these were provided with the best-performing colon segmentation method, which is part of the CoLesioNet of the present embodiments (see Table 1).

The method of the present embodiments was compared with several fully convolutional neural network (FCN) methods, including 2D U-Net, 3D U-Net, V-Net, and 3D nnU-Net. Table 2, below, summarizes the results using the FROC metric. In Table 2, sensitivity (%) is shown at various FPs/volume ratios.

TABLE 2

| | Sensitivity at FPs/volume | | | | |
|---|---|---|---|---|---|
| Method | 0.5 | 1 | 2 | 4 | Averag |
| 2D U-Net | 12.5 | 27.1 | 44.4 | 73.7 | 39.4 |
| 3D U-Net + Colon Seg | 41.8 | 60.4 | 70.6 | 76.4 | 62.3 |
| V-Net | 54.4 | 63.1 | 67.8 | 71.7 | 64.3 |
| 3D U-Net | 49.0 | 63.3 | 72.3 | 75.7 | 65.1 |
| 3D nnU-Net | 53.1 | 72.5 | 75.8 | 75.8 | 69.3 |
| CoLesioNet | 46.1 | 71.5 | 83.0 | 85.0 | 71.4 |
| CoLesioNet + Noisy Student | 57.0 | 74.2 | 86.7 | 87.5 | 76.4 |

The FCN approaches was evaluated in the following settings: (1) training the FCN model based on the lesion GT mask dataset; (2) training the FCN model based on both the lesion GT mask dataset and prior colon localization information (colon segmentation mask); (3) fine-tuning a 3D nnU-Net network pre-trained on the colon lesion challenge task [23]. For FCN methods that utilize the colon-phase output, the colon masks were fused to the input CT scan by concatenation. The method of the present embodiments was evaluated in two different settings: CoLesioNet supervised baseline, and CoLesioNet semi-supervised baseline (CoLesioNet+Noisy Student). The former network was trained only with the labeled patch dataset, and the latter network was trained with both the labeled patch dataset and the pseudo-labeled dataset.

As shown in Table 2, CoLesioNet achieved average sensitivity results superior to the compared approaches. This result demonstrates the difficulty of FCNs to generalize in the case of small datasets consisting of large 3D volumes, even when exploiting the colon mask. The patch-based method of the present embodiments outperforms the compared methods by a considerable margin in average sensitivity (71.4%). Specifically, in the fully-supervised setting, the CoLesioNet of the present embodiments excels over [22] by a large margin, especially at the 2 FP/scan and 4 FP/scan sensitivity levels, with improvements of 7.2% and 9.2%, respectively. The self-training strategy employed according to some embodiments of the present invention further boosts the fully-supervised baseline by 5% (from 71.4% to 76.4%), by leveraging the 550 unlabeled colon lesions volumes.

### IV Conclusion

This Example described a technique for colon segmentation and lesion detection in routine abdominal 3D CT scans. The technique was validated on a colon dataset and a colon lesion dataset, demonstrating that the CoLesioNet of the present embodiments outperforms other approaches in both tasks. This Example demonstrated that colon segmentation using a sparsely annotated dataset achieves good performance while requiring significantly less annotation effort from the radiologist, compared to pixel-wise labeling. This labeling technique may alleviate the annotation burden especially in large traversing organs like the colon, in which pixel-wise annotation is tedious. This Example also demonstrated high lesion detection performance indicating that the method of the present embodiments is useful for radiologists reviewing routine abdominal scans while not adding extra effort. Moreover, using the semi-supervised learning approach the supervised baseline was further boosted, demonstrating the advantage of SSL, for example, in small dataset conditions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] R. L. Siegel et al., “Colorectal cancer statistics, 2020,” CA: A Cancer Journal for Clinicians, vol. 70, no. 3, pp. 145-164, 2020.
[2] M. G. Keane and G. J. Johnson, “Early diagnosis improves survival in colorectal cancer,” Practitioner, vol. 256, no. 1753, pp. 15-18, 2012.
[3] American Cancer Society, “Colorectal Cancer Facts & FIGS. 2020-2022.” American Cancer Society Inc, pp. 1-32, 2020.
[4] H. Ojidu et al., “PTH-053 colonoscopy: the doctors' gold standard, but is it the patients'? a study of choice of colonic investigation made by fully informed patients,” Gut, vol. 66, no. Suppl 2, pp. A231-A231, 2017.
[5] S. Y. Kim, H. S. Kim, and H. J. Park, “Adverse events related to colonoscopy: Global trends and future challenges,” World Journal of Gastroenterology, vol. 25, no. 2, pp. 190-204, 2019.
[6] C. D. Johnson and P. J. Pickhardt, Diseases of the Colon and Rectum: CT Colonography. Milano: Springer Milan, 2014, pp. 56-58.
[7] L. Woodbridge and P. Wylie, “Current Issues in Computed Tomography Colonography,” Seminars in Ultrasound, CT and MRI, vol. 37, no. 4, pp. 331-338, 2016.
[8] S. Bynum, J. Davis, B. L. Green, and R. Katz, “Unwillingness to participate in colorectal cancer screening: Examining fears, attitudes, and medical mistrust in an ethnically diverse sample of adults 50 years and older,” American Journal of Health Promotion, vol. 26, pp. 295-300, 2012.
[9] N. J. Pelc, “Recent and Future Directions in CT Imaging,” Annals of Biomedical Engineering, vol. 42, no. 2, pp. 260-268, feb 2014.
[10] E. Klang et al., “Pitfalls in diagnosing colon cancer on abdominal CT,” Clinical Radiology, vol. 72, no. 10, pp. 858-863, 2017.
[11] J. Ker, L. Wang, J. Rao, and T. Lim, “Deep Learning Applications in Medical Image Analysis,” IEEE Access, vol. 6, pp. 9375-9389, 2018.
[12] D. Shen, G. Wu, and H. Suk, “Deep Learning in Medical Image Analysis,” Annual Review of Biomedical Engineering, vol. 19, no. 1, pp. 221-248, jun 2017.
[13] I. Pacal, D. Karaboga, A. Basturk, B. Akay, and U. Nalbantoglu, “A comprehensive review of deep learning in colon cancer,” Computers in Biology and Medicine, vol. 126, p. 104003, 2020.
[14] F. Milletari, N. Navab, and S. Ahmadi, “V-Net: Fully convolutional neural networks for volumetric medical image segmentation,” in Proc. Int. Conf. 3D Vis. (3DV), Stanford, CA, USA, 2016, pp. 565-571.
[15] Ö. Çiçek, A. Abdulkadir, S. Lienkamp, T. Brox, and O. Ronneberger, “3D U-Net: Learning dense volumetric segmentation from sparse an-notation,” in Proc. Int. Conf. Med. Image Comput. Comput.-Assist. Intervent. (MICCAI), Athens, Greece, 2016, pp. 424-432.
[16] X. Li, H. Chen, X. Qi, Q. Dou, C. W. Fu, and P. A. Heng, “H-DenseUNet: Hybrid densely connected UNet for liver and tumor seg-mentation from CT volumes,” IEEE Trans. Med. Imag., vol. 37, no. 12, pp. 2663-2674, 2018.
[17] K. Kamnitsas et al., “Efficient multi-scale 3D CNN with fully connected CRF for accurate brain colon lesion detection,” Medical Image Analysis, vol. 36, pp. 61-78, 2017.
[18] S. Chen, K. Ma, and Y. Zheng, “Med3D: Transfer learning for 3D medical image analysis,” arXiv: 1904.00625, 2019.
[19] A. M. Godkhindi and R. M. Gowda, “Automated detection of polyps in CT colonography images using deep learning algorithms in colon cancer diagnosis,” in Proc. Int. Conf. Energy Commun. Data Analytics Soft Comput. (ICECDS), Chennai, India, 2017, pp. 1722-1728.
[20] G. Urban et al., “Deep Learning Localizes and Identifies Polyps in Real Time With 96% Accuracy in Screening Colonoscopy,” Gastroenterology, vol. 155, no. 4, pp. 1069-1078.e8, 2018.
[21] J. Deng, W. Dong, R. Socher, L. Li, Kai Li, and Li Fei-Fei, “Imagenet: A large-scale hierarchical image database,” in Proc. IEEE Conf. Comput. Vis. Pattern Recognit, Miami, FL., USA, 2009, pp. 248-255.
[22] “nnU-Net: Self-adapting framework for U-Net-based medical image segmentation,” in Bildverarbeitung für die Medizin 2019. Wiesbaden, Germany: Springer Fachmedien Wiesbaden, 2019, pp. 22-22.
[23] A. L. Simpson et al., “A large annotated medical image dataset for the development and evaluation of segmentation algorithms,” arXiv, vol. abs/1902.09063, 2019.
[24] S. Li, C. Zhang, and X. He, “Shape-aware semi-supervised 3D semantic segmentation for medical images,” in Proc. Int. Conf. Med. Image Com-put. Comput.-Assist. Intervent. (MICCAI). Cham: Springer International Publishing, 2020, pp. 552-561.
[25] C. Baur, B. Wiestler, S. Albarqouni, and N. Navab, “Fusing unsupervised and supervised deep learning for white matter colon lesion detection,” in Proc. Int. Conf. Med. Imag. Deep Learn. (MIDL), vol. 102, London, United Kingdom, 2019, pp. 63-72.
[26] M. Golhar, T. L. Bobrow, M. P. Khoshknab, S. Jit, S. Ngamruengphong, and N. J. Durr, “Improving colonoscopy lesion classification using semi-supervised deep learning.” IEEE Access, vol. 9, pp. 631-640, 2021.
[27] Q. Xie, M. T. Luong. E. Hovy, and Q. V. Le, “Self-training with noisy student improves imagenet classification,” in Proc. IEEE Conf. Comput. Vis. Pattern Recognit. (CVPR), Seattle, WA. USA, 2020, pp. 10 684-10 695.
[28] A. Vaswani et al., “Attention is all you need,” in Proc. Adv. Neural Inf. Process. Syst. (NIPS), Red Hook, NY, USA, 2017, p. 6000-6010.
[29] X. Chen et al., “A dual-attention dilated residual network for liver lesion classification and localization on ct images,” in Proc. IEEE Int. Conf. Image Process. (ICIP), Taipei, Taiwan, 2019, pp. 235-239.
[30] A. Sinha and J. Dolz, “Multi-scale self-guided attention for medical im-age segmentation,” IEEE Journal of Biomedical and Health Informatics, vol. 25, no. 1, pp. 121-130, 2021.
[31] M. Li, W. Hsu, X. Xie, J. Cong, and W. Gao, “SACNN: Self-attention convolutional neural network for low-dose CT denoising with self-supervised perceptual loss network,” IEEE Trans. Med. Imag., vol. 39, no. 7, pp. 2289-2301, 2020.
[32] H. Zhang. I. Goodfellow, D. Metaxas, and A. Odena, “Self-attention generative adversarial networks,” in Proc. Int. Conf. Mach. Learn. (ICML), vol. 97, Long Beach, CA, USA, 2019, pp. 7354-7363.
[33] L. R. Dice, “Measures of the amount of ecologic association between species,” Ecology, vol. 26, no. 3, pp. 297-302, 1945.
[34] D. P. Kingma and J. Ba, “Adam: A method for stochastic optimization,” in Proc. Int. Conf. Learn. Representations (ICLR), San Diego, CA, USA, 2015.
[35] A. Paszke et al., “Pytorch: An imperative style, high-performance deep learning library,” in Proc. Adv. Neural Inf. Process. Syst. (NeurIPS), vol. 32, Vancouver, Canada, 2019.
[36] K. Yan, X. Wang. L. Lu, and R. M. Summers, “DeepLesion: automated mining of large-scale lesion annotations and universal lesion detection with deep learning.” Journal of Medical Imaging, vol. 5, no. 03, p. 1. 2018.
[37] K. Yan et al., “Learning from multiple datasets with heterogeneous and partial labels for universal lesion detection in CT.” IEEE Trans. Med. Imag., pp. 1-1, 2020.
[38] H. R. Roth et al., “Improving Computer-Aided Detection Using Convolutional Neural Networks and Random View Aggregation.” IEEE Trans. Med. Imag., vol. 35, no. 5. pp. 1170-1181, may 2016.

1. A method of analyzing an abdominal computed tomography (CT) scan, the method comprising:
applying a colon segmentation machine learning procedure to the CT scan, and receiving from said procedure an output indicative of a plurality of colon segments; and
feeding said output into a colon lesion detection machine learning procedure, and receiving from said colon lesion detection machine learning procedure an output indicative of presence of at least one pathology in the colon.

2. The method according to claim 1, wherein said output of said colon segmentation machine learning procedure comprises a colon segmentation binary mask.

3. The method according to claim 1, wherein said output of said colon lesion detection machine learning procedure comprises a colon lesion binary mask.

4. The method according to claim 1, comprising feeding the CT scan also into said colon lesion detection machine learning procedure.

5. (canceled)

6. The method according to claim 1, comprising defining a plurality of patches over the CT scan, wherein said colon segmentation machine learning procedure is applied separately to each patch.

7. (canceled)

8. The method according to claim 6, comprising feeding a position of each patch to said colon segmentation machine learning procedure.

9. (canceled)

10. The method according to claim 1, wherein said colon segmentation machine learning procedure comprises a convolutional neural network (CNN) having convolutional layers.

11. (canceled)

12. The method according to claim 8, wherein said colon segmentation machine learning procedure comprises a convolutional neural network (CNN) having convolutional layers and a fully connected layer receiving said position together with an output from said convolutional layers.

13. (canceled)

14. The method according to claim 1, comprising defining a plurality of patches over the CT scan, wherein said colon lesion detection machine learning procedure is applied separately to each patch.

15. (canceled)

16. The method according to claim 1, wherein said colon lesion detection machine learning procedure comprises a convolutional neural network (CNN) having convolutional layers.

17. (canceled)

18. The method according to claim 1, comprising acquiring said CT scan from a subject having non-empty and un-insufflated colon.

19. (canceled)

20. The method according to claim 18, comprising transmitting said CT scan to a remote location, wherein said applying and said feeding is executed by a computer at said remote location.

21. (canceled)

22. A computer software product, comprising a computer-readable medium in which program instructions are stored, which instructions, when read by a computer, cause the data processor to receive an abdominal computed tomography (CT) scan and to execute the method according to claim 1.

23. (canceled)

24. A system for analyzing an abdominal computed tomography (CT) scan, the system comprising:
a computer readable medium storing a trained colon segmentation machine learning procedure, and a trained colon lesion detection machine learning procedure; and
a computer, configured to access said computer readable medium, to apply said colon segmentation machine learning procedure to the CT scan, to receive from said procedure an output indicative of a plurality of colon segments, to feed said output into said colon lesion detection machine learning procedure, and to receive from said colon lesion detection machine learning procedure an output indicative of presence of at least one pathology in the colon.

25. The system according to claim 24, wherein said output of said colon segmentation machine learning procedure comprises a colon segmentation binary mask.

26. The system according to claim 24, wherein said output of said colon lesion detection machine learning procedure comprises a colon lesion binary mask.

27. The system according to claim 24, wherein said computer is configured to feed the CT scan also into said colon lesion detection machine learning procedure.

28. The system according to claim 24, wherein said computer is configured to defining a plurality of patches over the CT scan, wherein said colon segmentation machine learning procedure is applied separately to each patch.

29. The system according to claim 28, wherein said computer is configured to feed a position of each patch to said colon segmentation machine learning procedure.

30. The system according to claim 24, wherein said colon segmentation machine learning procedure comprises a convolutional neural network (CNN) having convolutional layers.

31. The system according to claim 29, wherein said colon segmentation machine learning procedure comprises a convolutional neural network (CNN) having convolutional layers and a fully connected layer receiving said position together with an output from said convolutional layers.

32. The system according to claim 24, wherein said computer is configured to define a plurality of patches over the CT scan, wherein said colon lesion detection machine learning procedure is applied separately to each patch.

33. The system according to claim 24, wherein said colon lesion detection machine learning procedure comprises a convolutional neural network (CNN) having convolutional layers.
